(19) United States
(12) Patent Application Publication
(10) Pub. No.: US 2022/0296421 A1
(43) Pub. Date: Sep. 22, 2022

(54) EYEWEAR HEARING PROTECTION SYSTEMS

(71) Applicants: Andrew Leland, Reno, NV (US); Austin Ledingham, Sheridan, WY (US)

(72) Inventors: Andrew Leland, Reno, NV (US); Austin Ledingham, Sheridan, WY (US)

(21) Appl. No.: 17/202,493

(22) Filed: Mar. 16, 2021

(51) Int. Cl. A61F 11/12; H04R 1/10; G02C 11/00

(52) U.S. Cl. CPC A61F 11/12; H04R 1/1016; G02C 11/00; H04R 2201/023; A61F 2210/009; A61F 2011/085

(57) ABSTRACT

The present disclosure describes eyewear hearing protection systems and eyewear attachable hearing protectors. In one example, an eyewear hearing protection system can include a lens portion that includes a lens to be positioned in front of an eye of a user. A head support can extend back from the lens portion. An earplug dock can be positioned or positionable on the head support. An earplug can be attractable to and dockable on the earplug dock. In another example, an eyewear attachable hearing protector can include an earplug dock including an attachment portion to attach to a head support of an eyewear, and an earplug that is attractable to and dockable on the earplug dock.

### RELATED APPLICATION

None.

### GOVERNMENT INTEREST

None.

### BACKGROUND

A variety of activities and occupations can involve exposure to sound at high intensity (volume) levels. In many cases, exposure to such loud sounds can be damaging to the human ear, resulting in partial or total hearing loss. Acute exposure to very loud sounds or prolonged, repeated exposure to moderately loud sounds can cause such hearing loss in the person performing the particular occupation or activity. For example, sounds with an intensity above about 85 decibels (dBA) can cause gradual hearing loss over time, after prolonged or repeated exposure. Sounds above about 120 decibels can cause acute damage to the human ear. Some occupations can involve repeated exposure to sounds at these intensity levels, such as dentistry, surgery, machining, construction, firearm operation, airport staff, factory work, and others. Hearing protection can be recommended for individuals participating in such activities.

### SUMMARY

The technology described herein includes eyewear hearing protection systems and eyewear attachable hearing protectors. In one example, an eyewear hearing protection system includes a lens portion that includes a lens to be positioned in front of an eye of a user. A head support extends back from the lens portion. An earplug dock is positioned or positionable on the head support. An earplug is attractable to and dockable on the earplug dock. In some examples, the earplug dock can include a magnet and the earplug can be attractable to the earplug dock by the magnet. In other examples, a self-retracting cord can be connected to the earplug and the earplug dock, and the earplug can be attractable to the earplug dock by the self-retracting cord.

In another example, an eyewear attachable hearing protector includes an earplug dock including an attachment portion that is configured to attach to a head support of an eyewear. An earplug can be attractable to and dockable on the earplug dock. Again, in some examples, the earplug dock can include a magnet and the earplug can be attractable to the earplug dock by the magnet. In other examples, a self-retracting cord can be connected to the earplug and the earplug dock, and the earplug can be attractable to the earplug dock by the self-retracting cord.

With the examples set forth in the Summary above, it is noted when describing the eyewear hearing protection systems and the eyewear attachable hearing protectors, individual or separate descriptions are considered applicable to one other, whether or not explicitly discussed in the context of a particular example or embodiment. For example, in discussing an earplug that can be included in an eyewear hearing protection system, this discussion can also be applicable to eyewear attachable hearing protectors, and vice versa.

There has thus been outlined, rather broadly, certain features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is a perspective view of an example eyewear hearing protection system in accordance with examples of the present disclosure.

FIG. 2 is a perspective view of another example eyewear hearing protection system in accordance with examples of the present disclosure.

FIG. 3A is a perspective view of yet another example eyewear hearing protection system in accordance with examples of the present disclosure.

FIG. 3B is a side cross-sectional view of the example of FIG. 3A.

FIG. 3C is a front cross-sectional view of the example of FIG. 3A.

FIG. 4A is a side cross-sectional view of an example earplug dock and an example earplug in accordance with examples of the present disclosure.

FIG. 4B is a perspective view of the example earplug dock and example earplug of FIG. 4A.

FIG. 5 is a perspective view of another example eyewear hearing protection system in accordance with examples of the present disclosure.

FIG. 6 is a side cross-sectional view of another example earplug dock and example earplug in accordance with examples of the present disclosure.

FIG. 7A is a perspective view of another example earplug dock in accordance with examples of the present disclosure.

FIG. 7B is a perspective view of an example eyewear attachable hearing protector including the example earplug dock of FIG. 7A.

FIG. 7C is another perspective view the example earplug dock of FIG. 7A.

These drawings are provided to illustrate various aspects of the invention and are not intended to be limiting of the scope in terms of dimensions, materials, configurations, arrangements or proportions unless otherwise limited by the claims.

### DETAILED DESCRIPTION

Although hearing protection may be recommended for many activities and occupations that involve exposure to loud sound or noise, many people fail to use hearing protection for a variety of reasons. In some cases, hearing protectors may be uncomfortable or cumbersome. Some activities may require removing hearing protectors for conversation and then replacing the hearing protectors to protect from noise. This can be inconvenient for the user of the hearing protectors and may result in the user forgoing hearing protection altogether. As an example, dentists often use noisy tools such as drills, suction tubes, and others. Noise generated by these tools can reach sufficient intensity levels to cause hearing damage as dentists are repeatedly exposed to the noise over time. Despite this possibility of hearing damage, many dentists do not use hearing protectors when using these tools. This may be due, at least in part, to the inconvenience of putting on hearing protectors and then removing the hearing protectors repeatedly.

The hearing protection systems described herein can provide a convenient way to protect the hearing of a user when the user is exposed to loud noise, while also allowing a convenient way to stow hearing protectors when not in use. The hearing protection systems can include eyewear, such as a pair of glasses, goggles, a face shield, or magnifying loupes. The eyewear can include a lens portion and a head support portion extending back from the lens portion. The lens portion can refer to the portion of the eyewear that includes a lens or lenses for a wearer to look through, which may be positioned in front of the eyes of the wearer. The head support can be any portion of the eyewear that holds the eyewear in place on the head of the wearer, such as temple arms that rest on the tops of the ears of the wearer or a head strap that wraps around the head of the wearer. The system can further include an earplug dock that can be on the head support or that can be positioned on the head support. In some examples, the earplug dock can be an integrated part of the head support or permanently attached to the head support. In other examples, the earplug dock can be an attachment that may be separate from the head support, but that can be attached to the head support and detached from the head support. Finally, the system can also include an earplug that is attractable to the earplug dock and dockable on the earplug dock.

As used herein, “attractable to” can refer to an ability of the earplug dock and the earplug to exert an attractive force from one to the other, which can tend to bring the earplug into contact with the earplug dock. In certain examples, the attractive force can originate from a magnet or multiple magnets. For example, the earplug dock can include a magnet and the earplug can include a magnet or a magnetically attractable material so that earplug is attracted to the earplug dock. In other examples, the attractive force can originate from a self-retracting cord that connects the earplug dock to the earplug. The self-retracting cord can pull the earplug to the earplug dock when the cord retracts. The term “dockable” can refer to the ability of the earplug to rest in a docked position in contact with the earplug dock. For example, an earplug can be docked on the earplug dock when the earplug is held in place on the dock by a magnet or by a retracted cord.

When the earplug dock is positioned on the eyewear head support, the earplug dock can be in close proximity to the ears of the wearer. Therefore, it can be convenient and easy for the wearer to remove the earplug from the earplug dock and insert the earplug into the ear of the wearer. Additionally, the wearer can easily remove the earplug from the ear and replace the earplug on the dock. As explained above, the earplug dock can exert an attractive force on the earplug that will pull the earplug into the docked position. This means that the wearer is not required to place the earplug precisely onto the earplug dock, which may be difficult when the wearer is not able to see the earplug dock or when the wearer may be focusing on another task. Instead, the wearer can merely move the earplug to the general vicinity of the earplug dock, and the attractive force between the earplug dock and the earplug can pull the earplug back into docked positioned. Thus, the wearer can easily return the earplug to the earplug dock without difficulty, even without seeing the earplug or the earplug dock.

While these example embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

The following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

### Definitions

It is noted that, as used in this specification and in the appended claims, the singular forms “a,” “an,” and “the” include plural referents unless the context clearly dictates otherwise. Thus, for example, reference to “a clamp” includes one or more of such features, reference to “an earplug” includes reference to one or more of such elements, and reference to “positioning” includes reference to one or more of such steps.

As used herein, the terms “about” and “approximately” are used to provide flexibility, such as to indicate, for example, that a given value in a numerical range endpoint may be “a little above” or “a little below” the endpoint. The degree of flexibility for a particular variable can be readily determined by one skilled in the art based on the context.

As used herein, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that, which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open ended term in this specification, like “comprising” or “including,” it is understood that direct support should be afforded also to “consisting essentially of” language as well as “consisting of” language as if stated explicitly and vice versa.

As used herein, comparative terms such as “increased,” “decreased,” “better,” “higher,” “lower,” and the like refer to a property of a device or component, that is measurably different from other devices or components, in a surrounding or adjacent area, in a single device or in multiple comparable devices, in a group or class, in multiple groups or classes, or as compared to the known state of the art. This applies both to the form and function of individual components in a device or process, as well as to such devices or processes as a whole.

As used herein, the term “substantially” refers to the complete or nearly complete extent or degree of an action, characteristic, property, state, structure, item, or result. The exact allowable degree of deviation from absolute completeness may in some cases depend on the specific context. However, the nearness of completion will generally be so as to have the same overall result as if absolute and total completion were obtained. The use of “substantially” is equally applicable when used in a negative connotation to refer to the complete or near complete lack of an action, characteristic, property, state, structure, item, or result.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Measurements, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein.

Accordingly, the scope of the invention should be determined solely by the appended claims and their legal equivalents, rather than by the descriptions and examples given herein. Reference will now be made to the exemplary embodiments illustrated, and specific language will be used herein to describe the same. It will nevertheless be understood that no limitation of the scope of the technology is thereby intended. Additional features and advantages of the technology will be apparent from the detailed description which follows, taken in conjunction with the accompanying drawings, which together illustrate, by way of example, features of the technology. Furthermore, various modifications and combinations can be derived from the present disclosure and illustrations, and as such, the figures should not be considered limiting.

### Eyewear Hearing Protection Systems

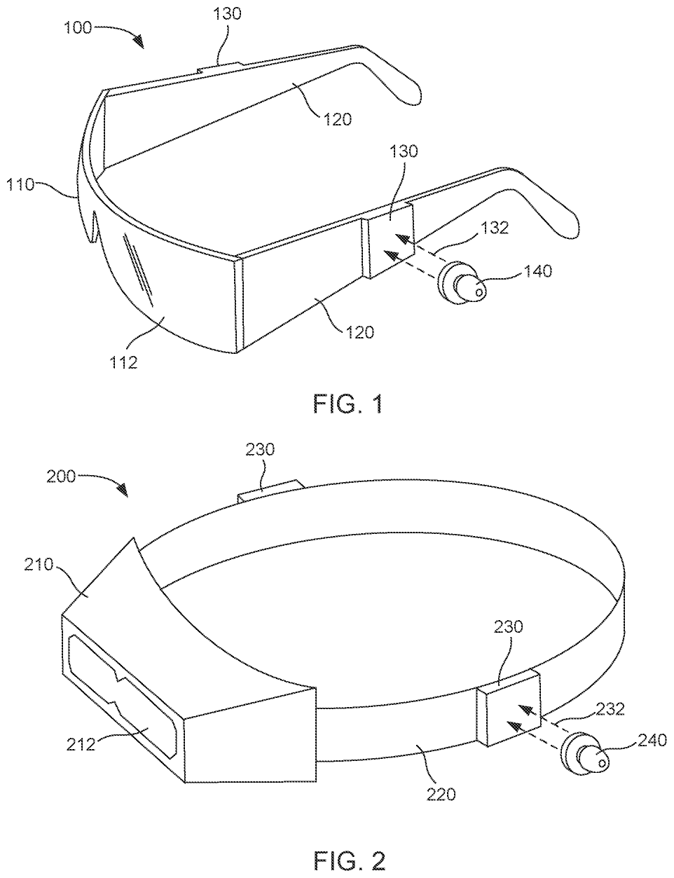

One example eyewear hearing protection system is shown in FIG. 1. The system 100 includes a lens portion 110, a head support 120, an earplug dock 130, and an earplug 140. The lens portion includes a lens 112. When the eyewear hearing protection system is worn by a user, the lens is positioned in front of an eye or both eyes of the user. The head support extends back from the lens portion. The earplug dock is positioned on the head support. The earplug is attractable to and dockable on the earplug dock. This figure shows dashed arrows 132 representing an attractive force that attracts the earplug toward the earplug dock. The attractive force can also hold the earplug in place on the earplug dock when the earplug is in a docked position.

The attractive force that attracts the earplug to the earplug dock can include a variety of attractive forces. In some examples, the attractive force can be a magnetic force. In a particular example, the earplug dock can include a magnet, and the earplug can be attracted by the magnet. The earplug can include a complementary magnet that is attracted to the magnet of the earplug dock. Alternatively, the earplug can include a paramagnetic material, such as steel or iron, that is attracted to the magnet. In another example, the earplug can include a magnet and the earplug dock can include a paramagnetic material that can be attracted to the magnet. As used herein, “magnet” refers to a permanent magnet or electromagnet that can create a magnetic field sufficient to attract the earplug. In some examples, the magnet can be a neodymium iron boron magnet, a samarium cobalt magnet, an alnico magnet, a ceramic magnet, a ferrite magnet, or a combination of these. Examples of eyewear hearing protection systems that include magnets are described in more detail below.

Another type of attractive force that can attract the earplug to the earplug dock is a retractable cord. A self-retracting cord can be connected to the earplug and the earplug dock. When the cord retracts, the earplug can move toward the earplug dock. The cord can be designed so that retracting the cord a sufficient amount will bring the earplug into contact with the earplug dock. This can be referred to as the docked position of the earplug on the earplug dock. As used herein, “self-retracting cord” refers to a cord that includes a retraction mechanism that can effectively shorten the length of cord between the earplug dock and the earplug. In some cases, the retraction mechanism can be located at the earplug dock (i.e., the retraction mechanism may be on the earplug dock, adjacent to the earplug dock, inside the earplug dock, etc.). In other cases, the retraction mechanism can be located at the earplug. In some examples, the retraction mechanism can include a spring-wound spool. The cord can wind around the spring-wound spool as the cord is retracted, and conversely the cord can unwind from the spool when the cord is extended. In particular examples, the retraction mechanism can include a spool lock that locks the spring-wound spool in an unwound position when the earplug is in the ear of the user. Examples of eyewear hearing protection systems that include self-retracting cords are described in more detail below.

The head support can include a feature or features that hold the eyewear hearing protection system on the head of a user. In the example shown in FIG. 1, the head support includes temple arms that extend back from the lens portion past the temples of the user. The temple arms are rigid supports that are shaped to rest on an ear of the user. The temple arms include ear hooks that grip the ears of the user. In this example, the earplug docks are located on the temple arms.

Other types of head supports can also be used, such as head straps, connected caps, helmets, face masks, or other types of head supports. FIG. 2 shows an example eyewear hearing protection system 200 that includes a head strap 220 as the head support. This example also includes a lens portion 210 that includes magnifying lenses 212. Accordingly, in this example, the eyewear portion of the system is used to magnify objects for the user. Earplug docks 230 are located on the head strap. Earplugs 240 are attracted to the earplug docks by an attractive force, illustrated as dashed arrows 232 similar to the previous example.

In the examples of FIG. 1 and FIG. 2, the earplug docks are integrated as a part of the head support. In the example of FIG. 1, the earplug docks are integrated parts of the temple arms. In the example of FIG. 2, the earplug docks are integrated parts of the head strap. Thus, in some examples, the earplug docks may be an integrated portion of the head support. In other examples, the earplug docks can be a discrete piece that is separate from the head support. Whether the earplug docks are an integrated portion of the head support or a separate piece from the head support, the earplug docks can be referred to as being positioned or positionable on the head support. In certain examples, an earplug dock can be a separate piece from the head support, but the earplug dock can be permanently attached to the head support by glue, screws, or another attachment method. In further examples, the earplug dock can be a separate piece from the head support that can be temporarily attached and detached if desired. Such an example earplug dock can attach to the head support using any suitable attachment device or method. In some examples, the earplug dock can attach to the head support using a clip, clamp, sleeve, hook and loop fasteners, snaps, buttons, ties, elastic bands, straps, or other devices or methods.

The location of the earplug docks on the head support can be anywhere on the head support. In some cases, if the earplug docks are attachable to the head support by a user, then the user can choose a desired location for the earplug docks. In some examples, the earplug docks can be located on the head support near the ear of the user. This can be convenient as the earplug is moved a small distance when the earplug is removed from the dock and placed in the ear, and vice versa. In some examples, the earplug dock can be located in front of the ear of the user, or in other words, in the direction nearer to the lens portion of the eyewear hearing protection system relative to the ear of the user. In other examples, the earplug dock can be located behind the ear of the user. In certain examples, the earplug dock can be located within a particular distance from the ear of the user, such as within about 1 inch, within about 2 inches, within about 3 inches, or within about 4 inches. If the earplug dock includes a self-retracting cord attached to the earplug, then the earplug dock can be located at a distance from the ear that is within the length of the self-retracting cord.

The earplug docks can be oriented in any direction. In examples shown in FIG. 1 and FIG. 2 the earplug docks are oriented facing outward from the head supports. In other words, the docking face of the earplug dock where the earplug can be docked is facing outward, or to the side with respect to the head of the user. In other examples, the earplug dock can have a docking face that faces backward, forward, upward, downward, at an angle, and so on.

Regarding the lens portion, the lens portion can include a lens or lenses that are positioned or positionable in front of an eye of a user. Depending on the type of eyewear, the lens portion can be always positioned in front of the eyes of the user when the eyewear is being worn, or the lens portion may be moveable so that the user can use the lenses at some times or move the lenses out of the way at other times. In some examples, the lens portion can have a hinge that allows the user to flip the lenses down when the user wishes to look through the lenses, and then flip the lenses back up to see without the lenses. A variety of different lens types can be included in the lens portion depending on the type of eyewear. In some examples, the eyewear can be protective glasses or goggles and the lens can be a protective lens that is designed to protect the eyes of the user. FIG. 1 shows an example of protective glasses with protective lenses. In other examples, the lenses can be magnifying lenses. FIG. 2 shows an example of magnifying goggles. In further examples, the lenses can be corrective lenses, such as the lenses in prescription eyeglasses. In certain examples, earplug docks can be attached onto corrective eyeglasses. Thus, the eyewear hearing protection systems described herein can be based on a user's corrective eyeglasses in some examples. In further examples, the eyewear can be a head-mounted display. The lenses of the lens portion can be display screens or display projectors used in such a head-mounted display.

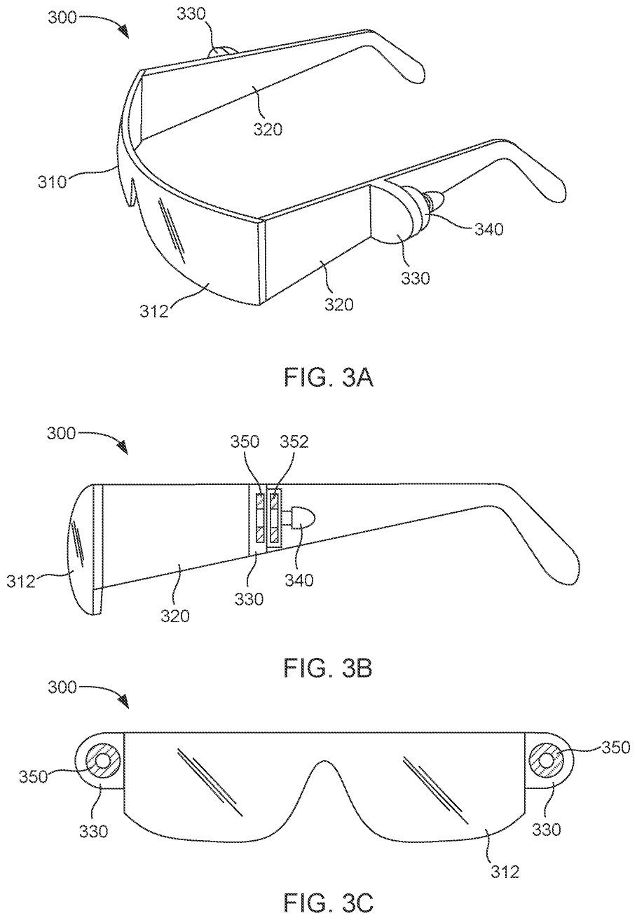

Example systems that include magnets will now be described in more detail. Another example eyewear protection system 300 is shown in FIGS. 3A-3C. FIG. 3A shows a perspective view of the eyewear protection system. The system includes a lens portion 310 with lenses 312 that can be positioned in front of the eyes of a user. A head support 320 extends back from the lens portion. In this example, the head support is made up of temple support arms that hook over the ears of the user. Earplug docks 330 extend out to the sides from the temple support arms. Earplugs 340 are attracted to the earplug docks by magnets. Thus, the earplugs can be docked by sticking against the earplug docks by magnetic force.

FIG. 3B shows a side cross-sectional view. In this figure, a first magnet 350 can be seen embedded in the earplug dock. The first magnet is a ring-shaped magnet. The cross-section of the ring-shaped magnet is shown from the side in this figure. A second ring-shaped magnet 352 is embedded in the earplug in this example. The first and second magnets can be oriented so that the earplug is attracted to the earplug dock (i.e., opposite poles of the first and second magnets can be oriented one toward another).

FIG. 3C shows a front cross-sectional view. This figure shows how the earplug docks 330 extend to the sides from the temple support arms. The first ring-shaped magnets 350 are shown embedded in the earplug docks.

As shown in FIGS. 3A-3C, the earplug docks can protrude from the head support in some examples. In certain examples, the earplug docks can protrude at an angle of about 90°, as shown in FIGS. 3A-3C. In other examples, the earplug docks can be oriented at a different angle with respect to the head support. Additionally, the earplug docks can have a docking surface that contacts the earplug when the earplug is docked. In some examples, the docking surface can be oriented as a vertical plane that protrudes at an angle from the head support, as shown in FIGS. 3A-3C. In some examples, the docking surface can face backward with respect to the line of sight of a user wearing the eyewear hearing protection system. In other examples, the docking surface can face forward, upward, downward, or at another direction at an angle between any of these directions. The docking surface can also be shaped to match the shape of the earplug, or at least partially match the shape of the earplug. For example, the docking surface can be rounded to match a circular or rounded shape of the earplug. In some examples, the docking surface can be rounded on outer edges as shown in FIGS. 3A-3C.

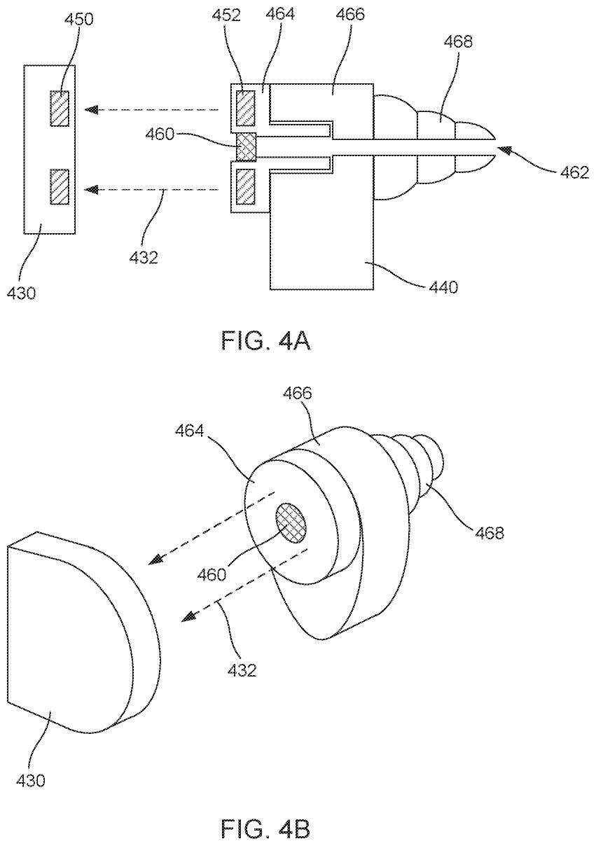

FIG. 4A and FIG. 4B show another example of an earplug dock 430 and an earplug 440. FIG. 4A is a cross-sectional side view. This view shows a first ring-shaped magnet 450 embedded in the earplug dock. A second ring-shaped magnet 452 is embedded in the earplug. The magnets attract each other through magnetic force, which is illustrated by two dashed arrows 432. The particular earplug shown in this example is designed to include a sound-attenuating filter 460. The sound attenuating filter can reduce the intensity of sound waves that pass through the filter, and in some cases the filter can be designed to reduce the intensity of sound at certain frequency ranges more than other frequency ranges. This type of earplug can be used in situations where the user desires to hear certain sounds more clearly, such as human voices, while blocking sounds at a particular frequency range, such as low frequencies or high frequencies. The earplug includes an open sound channel 462 and the sound-attenuating filter is placed in line with the sound channel to transmit filtered sound waves through the sound channel. The second ring-shaped magnet is positioned around the sound-attenuating filter so that the sound-attenuating filter is in the central opening of the second ring-shaped magnet. The second ring-shaped magnet and the sound-attenuating filter are both included in a filter portion 464, which is an insertable part that can be inserted into an earplug body portion 466. The earplug body portion is shaped to fit in the ear of the user and includes an ear canal-insertable portion 468.

Any suitable sound-attenuating filters can be used in the systems described herein. In some examples, the sound attenuating filter can have a noise reduction rating from about 5 decibels to about 35 decibels, or from about 5 decibels to about 27 decibels, or from about 9 decibels to about 27 decibels, or from about 20 decibels to about 35 decibels, or from about 5 decibels to about 10 decibels, or from about 5 decibels to about 15 decibels. Commercially available sound-attenuating filters include WESTONE® ER-9, ER-15, and ER-25 filters available from Westone Laboratories (USA); WHITE CAT™ Pro-Musician Acoustic Filters, Impact Acoustic Filters, and Living Acoustic Filters available from White Cat (New Zealand); ACS™ PRO10, PRO15, PRO17, PRO20, PRO26, PRO27, and PRO Impulse filters available from ACS (United Kingdom); and others.

Although ring-shaped magnets have been shown in the examples above, other types of magnets can be used in various examples. The magnet can have any convenient shape, such as a ring shape, cylindrical, square, bar shape, and so on. Multiple magnets can be used in some examples. One or multiple magnets can be included in the earplug dock. One or multiple magnets can also be used in the earplug. In certain examples, one or multiple magnets can be included in a filter portion that can be inserted into an earplug body. In other examples, one or multiple magnets can be included in the earplug body. In further examples, either the earplug dock or the earplug can include a paramagnetic material instead of a magnet. If the earplug dock or the earplug includes a paramagnetic material, and the other includes a magnet, then the magnet can attract the paramagnetic material.

The magnet can be embedded in the earplug dock or the earplug in some examples. As used herein, “embedded” refers to the magnet being at least partially surrounded by the material of the earplug dock or earplug, as opposed to being merely attached onto a surface of the earplug dock or earplug. In certain examples, the magnet can be embedded in such a way that the magnet is fully surrounded by the material of the earplug dock or earplug. In some examples, the earplug dock or earplug can be made of a polymer such as acrylonitrile butadiene styrene (ABS), polystyrene (PS), polylactic acid (PLA), polyurethane (PU), polyethylene (PE), polypropylene (PP), polyvinyl chloride (PVC), polycarbonate (PC), or others. When the magnet is fully embedded then the polymer material can surround the magnet on all sides so that the magnet is not visible to the user. The magnet can be embedded at a depth below the surface of the polymer material, where the depth is small enough that the magnetic force is still sufficient to attract the earplug to the earplug dock. In some examples, the magnet can be embedded at a depth from about 0.05 mm to about 5 mm below the surface of the earplug dock or earplug. In further examples, the depth can be from about 0.05 mm to about 2 mm, or from about 0.05 mm to about 1 mm, or from about 0.1 mm to about 5 mm, or from about 0.1 mm to about 2 mm, or from about 0.1 mm to about 1 mm, or from about 1 mm to about 5 mm, or from about 1 mm to about 3 mm. In alternative examples, the magnet can be embedded in such a way that one or more surfaces of the magnet are exposed and visible. In various examples, the magnet can be embedded in the earplug dock or earplug by insert molding, in which the magnet is placed in a mold and a polymer material is added into the mold around the magnet such that the magnet is embedded in the polymer material. In other examples, the earplug dock or earplug can be formed by additive manufacturing such as 3D printing. The magnet can be placed in an internal cavity at some point during the 3D printing process, and then the cavity can be closed using further 3D printing to embed the magnet within the part. In still other examples, the earplug dock or earplug can be formed as two or more pieces. The magnet can be assembled with the two or more pieces surrounding the magnet. In some examples, the two or more pieces can be designed to hold together, such as by pressure fitting. In other examples, the two or more pieces can be attached together by fusing, welding, screws, glue, or another attachment method. In still other examples, the earplug dock or earplug can have a depression or other feature for holding the magnet, and the magnet can be attached by pressure fitting, gluing, or another attachment method, such that the magnet is partially embedded and partially exposed.

The earplug can include an ear canal-insertable portion that can be inserted into an ear canal of the user. The ear canal-insertable portion can include a soft material that can conform to the ear canal and that can be comfortably inserted into the ear canal. In some examples, the soft material can be silicone, foam, or another material. In certain examples, the ear canal-insertable portion can include one or multiple silicone flanges. The example shown in FIGS. 4A and 4B includes a triple-flanged ear canal-insertable portion. In further examples, ear canal-insertable portion can have removable and replaceable tips. For example, the silicone flanged portion can be a removable part that can be replaced with a replacement silicone part of the same size or a different size. The ear canal-insertable portion can also include an open sound channel to allow sound through. As described above, in some examples the sound that is transmitted through the sound channel can be filtered by a sound-attenuating filter. In other examples, the ear plug may not include a sound channel. Instead, the earplug can be solid. This can increase the sound blocking ability of the earplug, but in some cases this can also decrease the clarity of sounds heard by the user and can make it difficult to have conversations, etc.

In addition to the ear canal-insertable portion, in some examples the earplug body can include a molded or moldable portion that can fit within the portion of the outer ear that is outside the ear canal. This portion can have a shape designed to fit in the average person's ear, or this portion can be custom molded for a specific user. Alternatively, this portion can include a moldable material that can allow a user to mold the earplug to fit the user's own ear.

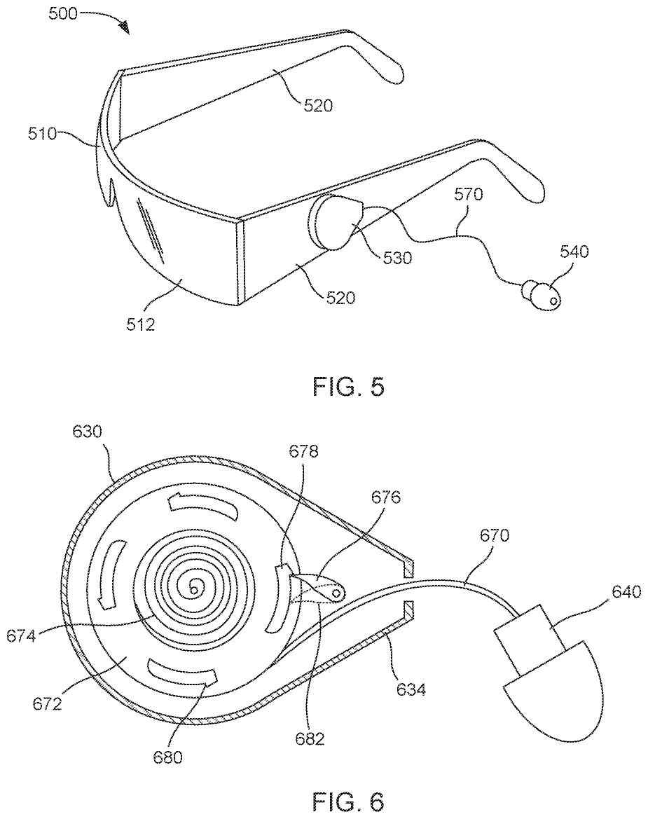

As mentioned above, the earplug can also be attractable to the earplug dock by a self-retracting cord. Examples that include self-retracting cords will now be described in more detail. FIG. 5 shows an example eyewear hearing protection system 500 that includes a lens portion 510 and a head support 520. The lens portion includes lenses 512. An earplug dock 530 is located on the head support. An earplug 540 is attractable to and dockable on the earplug dock. In this example, the earplug is attractable to the earplug by a self-retracting cord 570. The self-retracting cord is connected to the earplug and the earplug dock.

In various examples, the earplugs in systems that include a self-retracting cord can include similar components to the earplugs described above. Accordingly, in some examples, the earplugs can include earplug bodies, ear canal-insertable portions, sound-attenuating filters, filter portions that are insertable into the earplug body, and so on. The earplugs can be connected to the self-retracting cord through any suitable connection. For example, the earplug can include a hook, ring, loop, through-hole, or similar feature that the self-retracting cord can pass through. The self-retracting cord can include a loop at the end of the cord to loop through the connection feature on the earplug and retain the earplug. In other examples, and end of the self-retracting cord can be inserted into the earplug by molding the cord into the earplug, gluing the cord to the earplug, welding or fusing the cord to the earplug, or by another method.

In certain examples, the earplug can include a sound channel as described above. The sound channel can be aligned with a central portion of the earplug. The sound channel can be open to the air or the sound channel can be used with a sound-attenuating filter as described above. When a sound channel is included in a central portion of the earplug, the self-retracting cord can connect to the earplug at a connection point that is offset from the central portion of the earplug. Accordingly, in some examples the connection point can be in a peripheral portion of the earplug so that the connection point and the self-retracting cord do not block the sound channel.

The self-retracting cord can include cords that can retract by any suitable mechanism. In some examples, a retracting mechanism can be included in or on the earplug dock, in or on the earplug, or at a location along the cord between the earplug dock and the earplug. In some examples, the retraction mechanism can include a spring-wound spool. The cord can be wound around the spring-wound spool to retract the cord. The cord can then unwind from the spring-wound spool to lengthen the cord. In certain examples, the earplug can be placed in the ear of the user when the cord is unwound from the spool. When it is desired to dock the earplug, the earplug can be removed from the ear and the cord can wind around the spring-wound spool under the force of a spring in the spring-wound spool. Thus, in this example, the spring provides the attractive force that attracts the earplug to the earplug dock. The cord can retract until the earplug contacts the earplug dock, at which point the earplug can be in a docked position. In some examples, the earplug dock can have a docking surface that includes a hole through which the cord retracts. The docking surface can face in any direction. In the example shown in FIG. 5, the earplug dock has a docking surface facing backwards with respect to the line of sight of the user wearing the eyewear hearing protection system. The docking surface in this example is a small surface with a hole through which the cord retracts. In other examples, the docking surface can face outward, forward, upward, downward, or at an angle between any of these directions.

FIG. 6 shows a cross-sectional side view of an earplug dock 630 and an earplug 640 that are connected by a self-retracting cord 670. This figure shows components of the retraction mechanism for the self-retracting cord. The retraction mechanism is located within the earplug dock in this example. In particular, the earplug dock includes a spring-wound spool 672. A spring 674 provides rotational force that tends to turn the spool clockwise, to wind the cord around the spool. The earplug dock also includes a spool lock 676 that can lock the spring-wound in an unwound position. The spool lock can contact catches 678 on the spool. When the cord is unwound, the spool turns in the counter-clockwise direction and the catches can contact the spool lock and push the spool lock upward. If the user releases the cord while the spool lock is in contact with a catch, then the spool lock can be caught in a detent 680 at an upper end of the catch (the end of the catch having the detent can be described as upper as it upward when the eyewear hearing protection system of this example is being worn by a user). A slight tug on the cord can be sufficient to turn the spool until the spool clears the lower end of the catch. The spool lock can include its own spring (not shown) that can provide a force to return the spool lock to an intermediate position. If the user then releases the cord, the spool can turn clockwise and the bottom end of the catch can push the spool lock into a re-winding position 682 that is shown in dotted lines in FIG. 6. In this position, the spool lock does not prevent the spool from turning and the spool can rewind until the earplug is docked on the earplug dock. In this example, the components described above are contained within an earplug dock housing 634.

### Eyewear Attachable Hearing Protector

The present disclosure also describes eyewear attachable hearing protectors. The eyewear attachable hearing protectors can include an earplug dock and an earplug that is attractable to and dockable on the earplug dock. Additionally, the earplug dock can include an attachment portion that is configured to attach to a head support of an eyewear. In some examples, an eyewear attachable hearing protector can be attached to eyewear, and the eyewear and the eyewear attachable hearing protector together can make up an eyewear hearing protection system as described above.

In various examples, the eyewear attachable hearing protector can include any of the earplug docks and earplugs that are described above, including any of the features and components that are described above for the earplug docks and earplugs. However, the earplug dock of the eyewear attachable hearing protector can also include an attachment portion that is configured to attach to a head support of an eyewear. The attachment portion can use any suitable attachment mechanism to attach the earplug dock onto the head support of the eyewear. In some examples, the attachment portion can include a clamp, a clip, a sleeve, hook and loop fasteners, snaps, buttons, ties, elastic bands, straps, or others devices or methods. In certain examples, the attachment portion can be designed to be attached permanently to the head support of the eyewear. In other examples, the attachment portion can be designed to be removable from the head support of the eyewear.

In a particular example, the eyewear attachable hearing protector can have a clamp for clamping onto a head support of eyewear. The clamp can include an inside piece and an outside piece that clip together to sandwich the head support of the eyewear between the inside piece and the outside piece. As used herein, “inside piece” can refer to a piece that is placed on an inner surface of the head support, meaning between the head support and the head of the user. The “outside piece” refers to a piece that is placed on an outer surface of the head support, or the surface opposite from the head of the user. The inside piece and outside piece can clip together using a suitable attachment mechanism to sandwich the head support between the inside piece and the outside piece. In alternative examples, the clamp can include a top piece and a bottom piece, which can be clamped onto a top surface and a bottom surface of the head support, respectively. In some cases, the clamp can be designed to clamp onto temple supports such as the temple supports of eyeglasses or safety glasses. In other cases, the clamp can be designed to clamp onto a head strap.

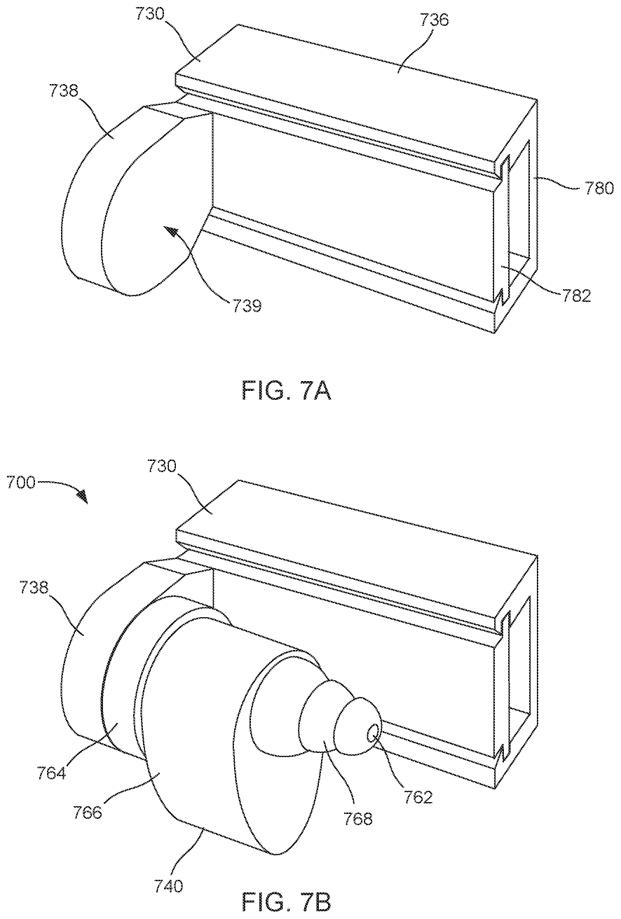

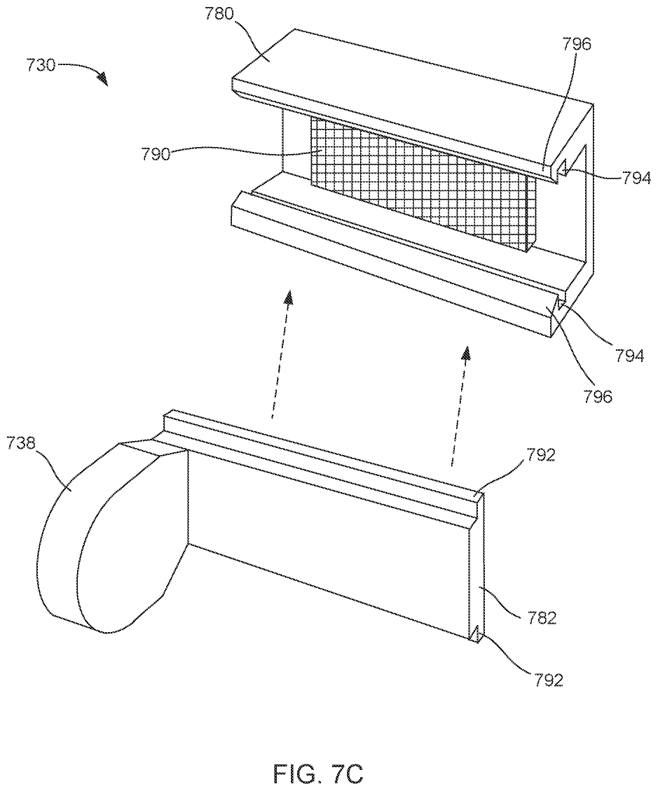

FIG. 7A shows an example earplug dock 730 of an example eyewear attachable hearing protector. The earplug dock includes an attachment portion 736 and a docking portion 738. The docking portion includes a docking surface 739, which is the surface that can contact the earplug when the earplug is docked. In this example, the attachment portion includes an inside piece 780 and an outside piece 782 that clip together. The inside piece and the outside piece can be clipped together with a temple support of eyeglasses or protective glasses between the inside piece and the outside piece. The docking portion is on the outside piece. Although not shown in the figure, the docking portion includes a ring-shaped magnet embedded beneath the docking surface, as described in some previous examples above.

FIG. 7B shows the earplug dock 730 assembled with an earplug 740 docked on the docking surface of the earplug dock. Together, the earplug dock and earplug make up an eyewear attachable hearing protector 700. The earplug can include any of the features described above with respect to the earplugs in eyewear hearing protection systems. In this example, the earplug includes a filter portion 764 that is inserted into an earplug body portion 766. The filter portion can include a sound-attenuating filter and an embedded magnet (not shown). The earplug body portion can include an ear canal-insertable portion 768. The earplug also includes an open sound channel 762 to allow filtered sound to pass through the earplug.

The earplug in this example can be easily detached (undocked) from the earplug dock by a user, and placed into the user's ear. The user can then remove the earplug from the ear and dock the earplug on the earplug dock. Docking the earplug can be easy and convenient since the earplug dock can be attached to the user's eyewear, which is close to the user's ear. Additionally, the user can dock the earplug easily even though the earplug dock may be outside the user's field of view, because the magnet in the earplug dock can attract the earplug toward the earplug dock. As long as the user brings the earplug close enough to the earplug dock for the magnets to provide a sufficient force, the magnets can pull the earplug into the docked position.

FIG. 7C shows the earplug dock 730 separated into an inside piece 780 and an outside piece 782. This figure demonstrates how the inside piece and the outside piece can clip together to clamp the earplug dock onto the head support of eyewear. This particular example is designed to clamp onto a temple support of eyeglasses, safety glasses, or the like. The temple support can be placed between the inside piece and the outside piece of the earplug dock and then the inside piece and outside piece can be clipped together. The inside piece and/or outside piece can also include a padding material 790 that can protect the temple support of the eyewear from being scratched or otherwise damaged. In some examples, the padding material can be a layer of soft, non-scratch material such as rubber, silicone, foam, or another such material.

A variety of clipping mechanisms can be used to clip the earplug dock onto the head support of the eyewear. In the example shown in FIG. 7C, the clipping mechanism includes tongues 792 on the sides of the outside piece and grooves 794 on the inside piece. When the inside piece and the outside piece clip together, the tongues fit into the grooves. The inside piece also includes sloped catches 796, which can allow the outside piece to be pressed into place until the tongues pass the sloped catches and fit into the grooves. In some examples, the material of the inside piece can be slightly flexible to allow the sloped catches to flex outward slightly when the outside piece is pressed into the opening between the sloped catches. Thus, although the material of the inside piece can be substantially rigid, the material can have a slight flexibility that is sufficient to allow the outside piece to clip to the inside piece. Alternatively, the outside piece can slide into place by aligning the tongues with the grooves and sliding the tongues into the grooves instead of pressing the tongues into the grooves. It should be noted that the clipping mechanism shown in FIG. 7C is one example, but a wide variety of other clipping or clamping mechanisms can be used to attach the earplug dock onto eyewear.

The dimensions of the eyewear attachable hearing protector are not particularly limited. In some examples, the earplug dock attachment portion can have a length (along the lengthwise direction of the eyewear head support) that will fit on the type of head support that the earplug dock is designed to attach to. In various examples, the earplug dock attachment portion can be shaped and sized to attach to a temple support of eyewear, or to a head strap, or to any other type of eyewear head support. In certain examples, the length of the earplug dock attachment portion can be from about 1 cm to about 10 cm, or from about 1 cm to about 8 cm, or from about 1 cm to about 5 cm, or from about 1 cm to about 3 cm, or from about 2 cm to about 5 cm, or from about 2 cm to about 4 cm. The attachment portion can have a thickness (from an inside surface to an outside surface) from about 5 mm to about 3 cm, or from about 5 mm to about 2 cm, or from about 5 mm to about 1.5 cm, or from about 5 mm to about 1 cm, or from about 1 cm to about 2 cm, or from about 1 cm to about 1.5 cm, in some examples. These thicknesses can be for the attachment portion, not counting the docking portion. In further examples, the docking portion can extend outward from the attachment portion. As shown in FIGS. 7A-7C, in some examples the docking portion can be oriented at a 90° angle with respect to the attachment portion. The docking surface of the docking portion can face backwards, forwards, upwards, downwards, outwards, or at an angle between any of these directions. In the example shown in FIGS. 7A-7C, the docking surface can face backwards or forwards depending on the direction that the earplug dock is attached to the temple support of the eyewear. The docking portion can be sized and shaped to accommodate the earplug. The size and shape of earplugs can vary. In some examples, the earplug can include a filter portion that includes an embedded magnet. The docking portion of the earplug can be sized and shaped to match or partially match the shape of the filter portion of the earplug. In some examples, the filter portion can have a diameter from about 1 cm to about 4 cm, or from about 1 cm to about 3 cm, or from about 1 cm to about 2 cm, or from about 2 cm to about 4 cm, or from about 2 cm to about 3 cm. Accordingly, the docking portion of the earplug dock can be sized and shaped to accommodate the filter portion of the earplug having any of these diameters.

In some examples, the inside piece and the outside piece of the attachment portion can be sized and shaped to accommodate a particular head support. However, different eyewear can have head supports of differing sizes and shapes. In some examples, the inside piece and the outside piece can be sized and shaped to accommodate a temple support of eyeglasses. The thickness of the temple support can vary between different individual pairs of eyeglasses. To allow the eyewear attachable hearing protector to be used with a variety of different eyewear, in some examples the eyewear attachable hearing protector can include a kit of multiple inside pieces that have different depths. These different inside pieces can be used to make the eyewear attachable hearing protector fit on eyewear having head supports of different thicknesses. A user can select the inside piece that has the appropriate depth to accommodate the user's own eyewear. As an example, the kit can include a first inside piece with a depth to accommodate a 1 mm to 2 mm-thick head support, a second inside piece with a depth to accommodate a 2 mm to 4 mm-thick head support, and a third inside piece with a depth to accommodate a 5 mm to 10 mm-thick head support. A variety of other depth combinations can also be used.

In further examples of eyewear attachable hearing protectors, the earplug dock and earplug can include any of the features, components, materials, and characteristics described above. For example, although the example eyewear attachable hearing protector shown above includes a magnet to attract the earplug, in other examples a self-retracting cord can be used to attract the earplug to the earplug dock. Any of the mechanisms and designs described for the eyewear hearing protection systems can also be used in the eyewear attachable hearing protectors.

The described features, structures, or characteristics may be combined in any suitable manner in one or more examples. In the preceding description numerous specific details were provided, such as examples of various configurations to provide a thorough understanding of examples of the described technology. One skilled in the relevant art will recognize, however, that the technology may be practiced without one or more of the specific details, or with other methods, components, devices, etc. In other instances, well-known structures or operations are not shown or described in detail to avoid obscuring aspects of the technology.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. An eyewear hearing protection system, comprising:
a lens portion comprising a lens to be positioned in front of an eye of a user;
a head support extending back from the lens portion;
an earplug dock positioned or positionable on the head support; and
an earplug that is attractable to and dockable on the earplug dock.

2. The eyewear hearing protection system of claim 1, wherein the earplug dock comprises a magnet, and the earplug is attractable to the earplug dock by the magnet.

3. The eyewear hearing protection system of claim 2, wherein the earplug comprises a second magnet that is attracted to the magnet of the earplug dock.

4. The eyewear hearing protection system of claim 3, wherein the second magnet is ring-shaped.

5. The eyewear hearing protection system of claim 4, wherein the earplug further comprises a sound-attenuating filter positioned at a central opening of the ring-shaped second magnet.

6. The eyewear hearing protection system of claim 5, wherein the earplug comprises an earplug body portion and a filter portion that is insertable into the earplug body portion, wherein the filter portion comprises the sound-attenuating filter and the ring-shaped second magnet embedded therein, and wherein the earplug body portion comprises an ear canal-insertable portion.

7. The eyewear hearing protection system of claim 4, wherein the earplug further comprises a sound channel aligned with a central opening of the ring-shaped second magnet.

8. The eyewear hearing protection system of claim 1, further comprising a self-retracting cord connected to the earplug and to the earplug dock, wherein the earplug is attractable to the earplug dock by the self-retracting cord.

9. The eyewear hearing protection system of claim 8, wherein the earplug further comprises a sound channel through a central portion of the earplug, wherein the self-retracting cord is connected to the earplug at a connection point that is offset from the central portion of the earplug.

10. The eyewear hearing protection system of claim 8, wherein the earplug dock comprises a spring-wound spool, wherein the self-retracting cord is wound around the spring-wound spool when the earplug is docked, and wherein the self-retracting cord unwinds from the spring-wound spool such that the earplug can be placed in an ear of the user.

11. The eyewear hearing protection system of claim 10, wherein the earplug dock further comprises a spool lock to lock the spring-wound spool in an unwound position when the earplug is in the ear of the user.

12. The eyewear hearing protection system of claim 1, wherein the lens comprises a protective lens, a corrective lens, a magnifying lens, or a combination thereof.

13. The eyewear hearing protection system of claim 1, wherein the head support comprises a rigid temple support shaped to rest on an ear of the user or a strap to wrap around the head of the user.

14. An eyewear attachable hearing protector, comprising:
an earplug dock comprising an attachment portion configured to attach to a head support of an eyewear; and
an earplug that is attractable to and dockable on the earplug dock.

15. The eyewear attachable hearing protector of claim 14, wherein the earplug dock comprises a magnet, and the earplug is attractable to the earplug dock by the magnet.

16. The eyewear attachable hearing protector of claim 15, wherein the earplug comprises a second magnet that is attracted to the magnet of the earplug dock, wherein the second magnet is ring-shaped and wherein the earplug further comprises a sound-attenuating filter positioned at a central opening of the ring-shaped second magnet.

17. The eyewear attachable hearing protector of claim 14, further comprising a self-retracting cord connected to the earplug and to the earplug dock, wherein the earplug is attractable to the earplug dock by the self-retracting cord.

18. The eyewear attachable hearing protector of claim 17, wherein the earplug dock comprises a spring-wound spool, wherein the self-retracting cord is wound around the spring-wound spool when the earplug is docked, and wherein the self-retracting cord unwinds from the spring-wound spool such that the earplug can be placed in an ear of the user.

19. The eyewear attachable hearing protector of claim 12, wherein the attachment portion comprises a clamp to clamp onto the head support of the eyewear, wherein the clamp comprises an inside piece and an outside piece that clip together to sandwich the head support of the eyewear between the inside piece and the outside piece.

20. The eyewear attachable hearing protector of claim 19, further comprising a kit of multiple inside pieces that clip together with the outside piece, wherein the inside pieces have different depths to accommodate head supports of different thicknesses.
